(19) United States
(12) Patent Application Publication
(10) Pub. No.: US 2026/0335112 A1
(43) Pub. Date: Oct. 29, 2026

(54) STENT DELIVERY SYSTEM

(71) Applicant: BOSTON SCIENTIFIC SCIMED, INC., Maple Grove, MN (US)

(72) Inventors: Jacob Arlandson, Mahtomedi, MN (US); Peter L. Dayton, Brookline, MA (US); Mark P. Olson, St. Anthony, MN (US); Gene T. Storbeck, Millville, MA (US)

(73) Assignee: BOSTON SCIENTIFIC SCIMED, INC., Maple Grove, MN (US)

(21) Appl. No.: 19/660,861

(22) Filed: Apr. 28, 2026

Related U.S. Application Data
(60) Provisional application No. 63/983,803, filed on Feb. 16, 2026.
(60) Provisional application No. 63/796,865, filed on Apr. 29, 2025.

(51) Int. Cl. A61F 2/966

(52) U.S. Cl. CPC A61F 2/966

(57) ABSTRACT

Example stent delivery systems are disclosed. An example stent delivery system includes a handle, a catheter shaft extending distally from the handle, the catheter shaft including a proximal outer shaft. Further, the stent delivery system includes an inner rail coupled to the proximal outer shaft and a distal inner shaft coupled to the inner rail and a collar disposed along and translatable relative to the inner rail, the collar defining a first lumen configured to permit the inner rail to extend therethrough. Further, the stent delivery system includes a radially expandable stent disposed along a portion of the distal inner shaft. Further, the first lumen has a first cross-sectional shape which mates with a second cross-sectional shape of the inner rail such that relative rotational movement between the collar and the inner rail is inhibited while axial translation of the collar relative to the inner rail is permitted.

### CROSS REFERENCE TO RELATED APPLICATIONS

The present application claims the benefit of U.S. Provisional Patent Application Serial No. 63/983,803, filed Feb. 16, 2026 and U.S. Provisional Patent Application Serial No. 63/796,865, filed Apr. 29, 2025, the disclosures of which are incorporated herein by reference.

### TECHNICAL FIELD

The present disclosure generally relates to deployment of self-expanding stents in co-axial stent delivery systems. More specifically, the present disclosure is related to improving accuracy of stent placement and deployment by utilizing an internal actuation mechanism.

### BACKGROUND

In a conventional co-axial stent deployment system, a catheter shaft, which may be inserted through the working channel of an endoscope in some instances, may include an outer shaft slidably disposed over an inner shaft. To deploy a self-expanding stent therefrom, the outer shaft may be withdrawn proximally relative to the inner shaft to expose the stent from the distal end of the outer shaft, thereby allowing the stent to radially expand from a radially constrained configuration within the outer shaft.

In some co-axial stent delivery systems, stored tension in the system may be released during deployment, which may result in unintended forward movement of the inner shaft and stent. This unintended axial motion has the potential to affect placement accuracy and may require a user to counteract the forward movement during deployment.

Accordingly, it may be desirable to configure stent delivery systems to reduce or limit unintended forward movement of the inner shaft during stent deployment. Systems and methods configured to reduce or limit unintended forward movement of the inner shaft during stent deployment are disclosed herein.

### BRIEF SUMMARY

In an example, a stent delivery system includes a handle, a catheter shaft extending distally from the handle, the catheter shaft including a proximal outer shaft. Further, the stent delivery system includes an inner rail coupled to the proximal outer shaft and a distal inner shaft coupled to the inner rail and a collar disposed along and translatable relative to the inner rail, the collar defining a first lumen configured to permit the inner rail to extend therethrough. Further, the stent delivery system includes a radially expandable stent disposed along a portion of the distal inner shaft. Further, the first lumen has a first cross-sectional shape which mates with a second cross-sectional shape of the inner rail such that relative rotational movement between the collar and the inner rail is inhibited while axial translation of the collar relative to the inner rail is permitted.

In addition or alternatively to any example described herein, wherein the first cross-sectional shape and the second cross-sectional shape are non-circular.

In addition or alternatively to any example described herein, wherein the first cross-sectional shape and the second cross-sectional shape are semi-circular.

In addition or alternatively to any example described herein, wherein the first cross-sectional shape and the second cross-sectional shape include at least one flat surface.

In addition or alternatively to any example described herein, wherein the first cross-sectional shape and the second cross-sectional shape include a first lobe, a second lobe and a valley separating the first lobe and the second lobe.

In addition or alternatively to any example described herein, wherein the inner rail includes a guidewire lumen which is in communication with a guidewire lumen of the proximal outer shaft.

In addition or alternatively to any example described herein, further comprising a retractable sleeve attached to the collar.

In addition or alternatively to any example described herein, wherein the stent is radially constrained between the distal inner shaft and the retractable sleeve in a delivery configuration.

In addition or alternatively to any example described herein, further comprising an actuation shaft coupled to the collar, wherein actuation of the actuation shaft axially translates both the collar and the retractable sleeve relative to the inner rail.

In addition or alternatively to any example described herein, wherein the collar includes an actuation shaft lumen configured to permit at least a portion of the actuation shaft to extend therein.

In addition or alternatively to any example described herein, wherein the axial translation of the retractable sleeve permits deployment of the stent.

In addition or alternatively to any example described herein, wherein the mating cross-sectional shape of the first lumen and the inner rail inhibits twisting of the actuation shaft with the inner rail.

In addition or alternatively to any example described herein, wherein the retractable sleeve includes a braided structure including a first wire extending in a first helical direction along a length of the retractable sleeve, and wherein the retractable sleeve further includes a polymer filament wound in a second helical direction opposite the first helical direction, and wherein the first wire and the polymer filament are embedded within a base material used to construct the retractable sleeve.

In addition or alternatively to any example described herein, wherein the polymer filament has a refractive index similar to a refractive index of the base material.

Another example stent delivery system includes a handle, a catheter shaft extending distally from the handle, the catheter shaft including a proximal outer shaft. Further, the stent delivery system includes an inner rail coupled to the proximal outer shaft and extending distally therefrom, and a distal inner shaft coupled to the inner rail and extending distally therefrom. Further, the stent delivery system includes a collar disposed along and axially translatable relative to the inner rail, the collar defining a first lumen configured to permit the inner rail to extend therethrough. Further, the stent delivery system includes a retractable sleeve attached to the collar, the retractable sleeve including a base material and at least one reinforcing filament having a refractive index similar to a refractive index of the base material. Further, the stent delivery system includes a radially expandable stent positioned between the retractable sleeve and the distal inner shaft in a delivery configuration. Further, the first lumen has a first cross-sectional shape which mates with a second cross-sectional shape of the inner rail such that relative rotational movement between the collar and the inner rail is inhibited while axial translation of the collar relative to the inner rail is permitted.

In addition or alternatively to any example described herein, wherein the first cross-sectional shape and the second cross-sectional shape are non-circular.

In addition or alternatively to any example described herein, wherein the retractable sleeve includes at least one wire interwoven with the at least one reinforcing filament, wherein the at least one wire extends in a first helical direction and the at least one reinforcing filament extends in a second helical direction, wherein the second helical direction is opposite the first helical direction.

In addition or alternatively to any example described herein, wherein the at least one wire is formed of stainless steel, the at least one reinforcing filament is formed of polyamide, and the base material is a polyether block amide or a thermoplastic polyurethane.

Another example stent delivery system includes a handle and a catheter shaft extending distally from the handle, the catheter shaft including a proximal outer shaft. Further, the stent delivery system includes an inner rail fixed relative to the proximal outer shaft and extending distally therefrom, and a distal inner shaft fixed relative to the inner rail and extending distally therefrom. Further, the stent delivery system includes a collar disposed along and axially translatable relative to the inner rail, the collar defining a first lumen configured to permit the inner rail to extend therethrough. Further, the stent delivery system includes a retractable sleeve attached to the collar, the retractable sleeve including a base material and at least one reinforcing filament. Further, the stent delivery system includes a radially expandable stent positioned between the retractable sleeve and the distal inner shaft in a delivery configuration and an actuation shaft including a first end coupled to the handle and a second end coupled to the collar, wherein actuation of the actuation shaft is configured to axially translate the retractable sleeve in a distal-to-proximal direction to deploy the stent. Further, the first lumen has a first cross-sectional shape which mates with a second cross-sectional shape of the inner rail such that relative rotational movement between the collar and the inner rail is inhibited while axial translation of the collar relative to the inner rail is permitted.

In addition or alternatively to any example described herein, wherein the first cross-sectional shape and the second cross-sectional shape are non-circular.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of an exemplary stent delivery system.

FIG. 2 is a cross-sectional view of a portion of the exemplary stent delivery system of FIG. 1 taken along line 2-2.

FIG. 3A is a cross-sectional view of a portion of the exemplary stent delivery system of FIG. 1 taken along line 3-3.

FIG. 3B is a cross-sectional view of a portion of the exemplary stent delivery system of FIG. 1 taken along line 3-3.

FIG. 4 is a partial cross-sectional side view of the exemplary stent delivery system of FIG. 1 after deployment of the stent.

FIG. 5A is a perspective view of a portion of the exemplary stent delivery system of FIG. 1.

FIG. 5B is a perspective view of a portion of the exemplary stent delivery system of FIG. 1.

FIG. 6 illustrates a portion of a stent delivery system in accordance with another embodiment of the subject matter disclosed herein.

FIG. 6A is a cross-sectional view of a portion of the exemplary stent delivery system of FIG. 6 taken along line 6A-6A.

FIG. 6B is a cross-sectional view of a portion of the exemplary stent delivery system of FIG. 6 taken along line 6B-6B.

FIG. 7 illustrates the portion of the stent delivery system of FIG. 6 after deployment of the stent.

FIG. 7A is a cross-sectional view of a portion of the exemplary stent delivery system of FIG. 7 taken along line 7A-7A.

FIG. 8 is a perspective view of a portion of an alternative construction of the exemplary stent delivery system.

FIG. 8A is a cross-sectional view along line 8A-8A of FIG. 8.

FIG. 9 illustrates a portion of the exemplary stent delivery system of FIG. 1.

FIG. 10 illustrates a cross-sectional view of the portion of the exemplary stent delivery system shown in FIG. 9.

FIG. 11 illustrates a portion of the exemplary stent delivery system of FIG. 1.

FIG. 12 illustrates a cross-sectional view of the portion of the exemplary stent delivery system shown in FIG. 11.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

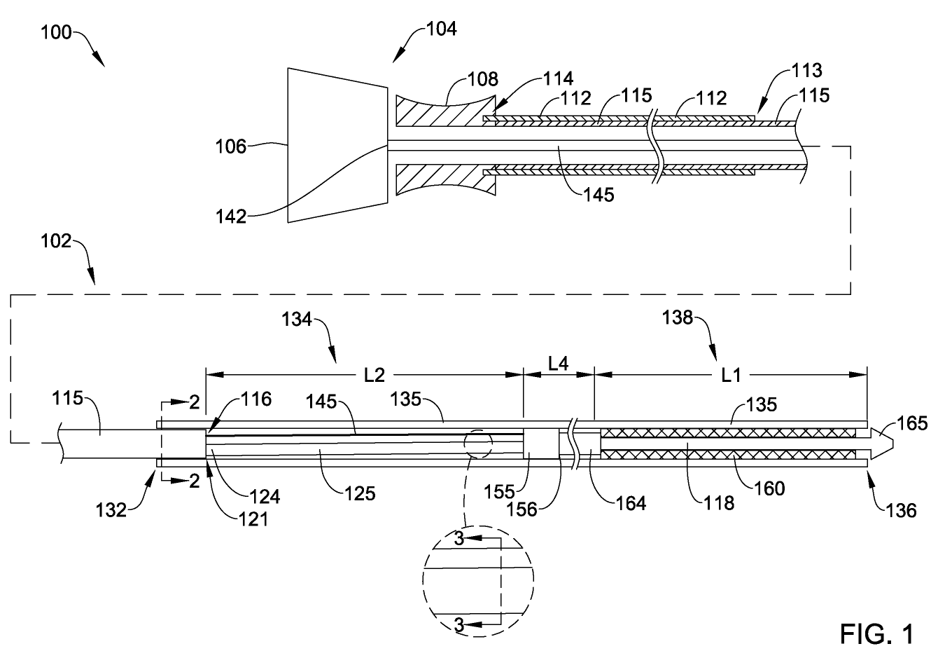

FIG. 1 shows a side view of a stent delivery system 100 with some components shown in cross-section. Stent delivery system 100 includes a handle assembly 104 and a catheter shaft 102 extending distally from the handle assembly 104, to deliver and deploy a radially expandable stent 160 in a body lumen of a patient. The catheter shaft 102 may include a proximal outer shaft 115, an outer reinforcing tube 112, an inner rail 125, a retractable sleeve 135, an actuation shaft 145, a distal inner shaft 118, and a collar 155.

To operate the stent delivery system 100, the handle assembly 104 includes a first, actuatable handle 106 and a second, stationary handle 108. The first, actuatable handle 106 may be actuated relative to the second stationary handle 108 to release the stent 160 from a delivery configuration to a deployment configuration. In the embodiment illustrated in FIG. 1 the first, actuatable handle 106 is positioned proximal to the second, stationary handle 108.

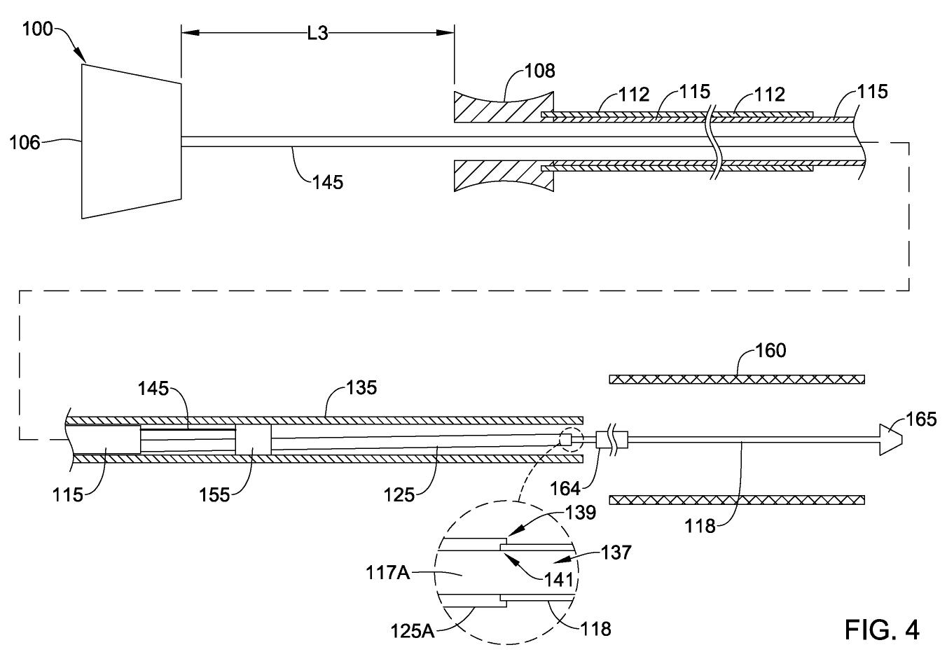

The proximal outer shaft 115 is fixed in position and secured to the second, stationary handle 108, such as fixed within or at an end of the second, stationary handle 108 of the handle assembly 104. The proximal outer shaft 115 may be secured to the second, stationary handle 108 and extend distally therefrom. Additionally, a proximal end region 124 of the inner rail 125 may be fixedly attached or secured to the proximal outer shaft 115, such as secured to a distal end region of the proximal outer shaft 115. As shown in FIG. 4, the inner rail 125 may extend distally from the distal end of the proximal outer shaft 115 and be attached to a proximal end region of the distal inner shaft 118. The distal inner shaft 118 may be fixedly attached or secured to the distal end region of the inner rail 125 and extend distally from the distal end of the inner rail 125.

FIG. 1 further illustrates an outer reinforcing tube 112 positioned along a portion of the proximal outer shaft 115. In some examples, a proximal end region 114 of the outer reinforcing tube 112 may extend into and be fixedly attached to the second, stationary handle 108. In some examples, the proximal end region 114 of the outer reinforcing tube 112 may extend into the handle 108 about 12 mm to about 14 mm. The outer reinforcing tube 112 may be fixedly attached to the outer surface of the proximal outer shaft 115 such that the outer reinforcing tube 112 is not axially translatable relative to the proximal outer shaft 115. In other words, the outer reinforcing tube 112 may be fixed relative to the proximal outer shaft 115 and the second, stationary handle 108. FIG. 1 illustrates the outer reinforcing tube 112 extending distally from the stationary handle 108 to a position in which the distal end 113 of the outer reinforcing tube 112 is located proximal of the distal end of the proximal outer shaft 115. Thus, the proximal outer shaft 115 may extend distally beyond the distal end 113 of the outer reinforcing tube 112. In some examples, the length of the outer reinforcing tube 112 may be about 102 centimeters (cm) to about 106 cm.

Further, in some examples, the inner diameter of the outer reinforcing tube 112 may be about 2.10 millimeters (mm) to about 3.0 mm and the wall thickness of the outer reinforcing tube 112 may be about 0.07 mm to about 0.40 mm. The outer reinforcing tube 112 may be formed of a polymeric material, such as polyetheretherketone (PEEK) or a similar material. It can be appreciated that the outer reinforcing tube 112 may provide a rigid, kink-resistant grip structure for a user to grasp, thereby preventing compression of the proximal outer shaft 115 and reducing the risk of pinching the actuation shaft 145 slidably extending within the proximal outer tube 115, which could otherwise lead to increased deployment forces.

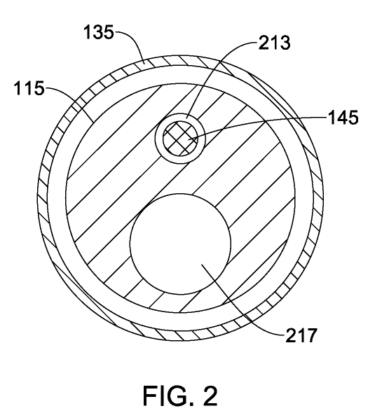

Referring to FIG. 2, it can be seen in the cross-sectional illustration that the proximal outer shaft 115 may include an actuation shaft lumen 213 and a guidewire lumen 217. In some instances, the proximal outer shaft 115 may be a multi-lumen tubular member formed of a monolithic construction defining both the actuation shaft lumen 213 and the guidewire lumen 217. Other shaft configurations are also contemplated. Although the proximal outer shaft 115 is illustrated with two lumens, additional lumens may be present.

FIG. 1 illustrates that the stent delivery system 100 may be an over-the-wire stent delivery system, whereby the guidewire lumen 217 may extend longitudinally from the proximal end of the proximal outer shaft to the distal end of distal tip 165. In this type of stent delivery system 100, the user may initially place the guidewire in the patient, then track the catheter shaft 102 (with the stent 160 loaded therein) over the guidewire to position the catheter shaft 102 adjacent a target tissue site. However, in other examples, the stent delivery system 100 may be configured as a rapid exchange stent delivery system in which the proximal outer shaft 115 includes a guidewire port located distal of the proximal end of the proximal outer shaft 115 that is in communication with the guidewire lumen 217 to allow a guidewire to exit the guidewire lumen 217 and extend along an exterior of the proximal outer shaft 115 proximal of the guidewire port. In some instances the guidewire port may be located between 30 centimeters (cm) and 60 cm from the distal tip 165, or about 40 cm from the distal tip 165.

A proximal end 142 of the actuation shaft 145 may be fixedly coupled or secured to the first, actuatable handle 106 and extend distally therefrom. For example, the actuation shaft 145 may be secured to the first, actuatable handle 106 and extend distally therefrom through the actuation shaft lumen 213 (shown in FIG. 2).

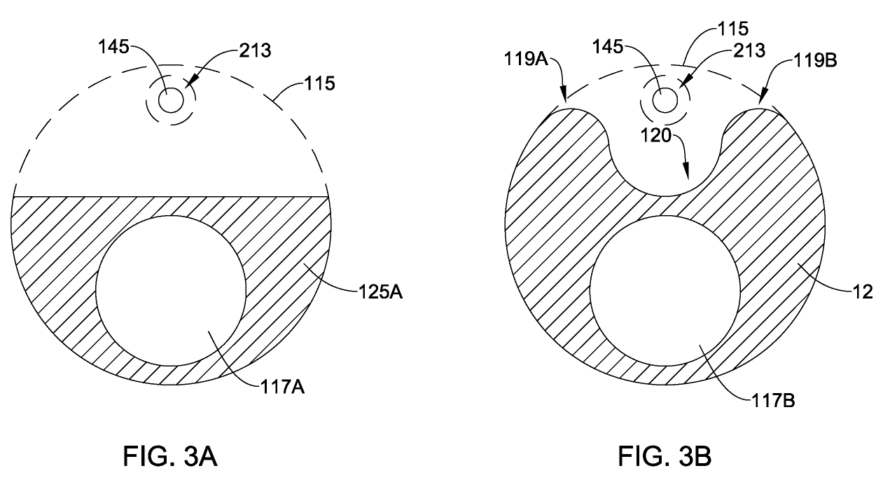

Additionally, in some examples the proximal end region 124 of the inner rail 125 may be coupled (e.g., fixedly attached) to the proximal outer shaft 115 such that an inner guidewire lumen 117A, 117B (examples shown in the cross-sectional illustration of FIGS. 3A, 3B, respectively) of the inner rail 125 may be in communication (e.g., axially aligned) with the guidewire lumen 217 of the proximal outer shaft 115 such that a guidewire may pass uninterrupted from the guidewire lumen 217 of the proximal outer shaft 115 to the guidewire lumen 117A, 117B of the inner rail 125.

Referring to FIG. 1, the retractable sleeve 135, which may be a tubular member, is illustrated in a delivery configuration (i.e., prior to stent deployment). The retractable sleeve 135 may be configured to surround the inner rail 125 and/or the distal inner shaft 118 and retain the stent 160 in a radially contracted, delivery configuration. When the retractable sleeve 135 is in the delivery configuration, the stent 160 may surround the distal inner shaft 118 and may be radially constrained by the distal sleeve region 138 of the retractable sleeve 135.

Among other considerations later discussed, the length of the retractable sleeve 135 may be chosen to ensure that the proximal end 132 of the retractable sleeve 135 remains in a working channel of an endoscope throughout operation of the stent delivery system 100 in order to prevent interference with the elevator of the endoscope.

The retractable sleeve 135 may include two regions, the distal sleeve region 138 and a proximal sleeve region 134 extending proximal of the distal sleeve region 138, together both which may surround the inner rail 125 and/or the distal inner shaft 118. In other words, the inner rail 125 may extend within the lumen of the proximal sleeve region 134 and the distal inner shaft 118 may extend within the lumen of the distal sleeve region 138. Further, a collar 155 may be secured (e.g., non-movably secured) or otherwise fixed to the retractable sleeve 135 along the proximal sleeve region 134 and/or the distal sleeve region 138. The collar 155 may be secured within the retractable sleeve 135 such that the tubular member of the retractable sleeve 135 surrounds the collar 155 with the proximal sleeve region 134 extending proximal of the collar 155 and the distal sleeve region 138 extending distal of the collar 155. Furthermore, the length of the retractable sleeve 135 may be chosen to ensure that the collar 155 remains within the working channel of the endoscope (e.g., proximal of the articulation zone of the endoscope) during a medical procedure. In other words, the collar 155 may be arranged at a sufficient length proximal of the stent 160 during delivery and deployment of the stent 160 such that the collar 155 remains located within the working channel of the endoscope throughout the medical procedure (i.e., throughout both delivery and deployment of the stent 160).

The distal sleeve region 138 may define a stent encapsulation pocket along length L1 of the retractable sleeve 135, where the stent 160 is radially constrained between an inner surface of the distal sleeve region 138 and an outer surface of the distal inner shaft 118. Length L1 may have a length sufficient to accommodate the full length of the stent 160 in its radially constrained configuration during delivery of the stent 160.

In some embodiments, the stent delivery system 100 may include a stent retention bumper or retainer 164, or a plurality of stent retention bumpers or retainers 164 fixedly (e.g., non-moveably) attached to an outer surface of the distal inner shaft 118. In some examples, length L1 of the stent encapsulation pocket may be defined as the length between the stent retention bumper or retainer 164 and the distal tip 165. The stent retention bumper or retainer 164 may be any desired shape configured to engage a portion of the stent 160 (e.g., engage or contact the proximal end of the stent 160) when radially constrained. In some instances, the stent retention bumper or retainer 164 may be a tubular (e.g., cylindrical) structure extending circumferentially around a portion of the distal inner shaft 118 and having a larger outer diameter than the outer diameter of the distal inner shaft 118. In other words, the stent retention bumper or retainer 164 may extend radially away from a central longitudinal axis of the distal inner shaft 118. In some embodiments (not illustrated), the stent delivery system 100 may include two stent retention bumpers 164, a distal stent retention bumper 164 located proximate to the distal end of the stent 160 and a proximal stent retention bumper 164 located proximate to the proximal end of the stent 160, to define the length L1 of the stent encapsulation pocket therebetween.

The stent retention bumper(s) or retainer(s) 164 may be configured to keep the longitudinal position of the stent 160 relative to the distal inner shaft 118 steady, preventing axial movement the stent 160 from following the movement of the retractable sleeve 135 as the retractable sleeve 135 is withdrawn proximally as the retractable sleeve 135 transitions into a deployment configuration for releasing the stent 160 (see FIG. 4). Accordingly, the stent retention bumper(s) or retainer(s) 164 prevents the stent 160 from following or responding to movements of the handle assembly 104 and/or the catheter shaft 102 prior to full deployment, further ensuring that the stent 160 is deployed on-target. In embodiments that include stent retention bumpers or retainers 164, the distal end 136 of the retractable sleeve 135 may be located distal to the stent retention bumpers or retainers 164 in the delivery configuration.

The distance between the distal end 116 of the proximal outer shaft 115 and the proximal end of the collar 155 has a length L2 in the delivery configuration in which the stent 160 is radially constrained within the distal sleeve region 138. Length L2 has a sufficient length to permit the proximal sleeve region 134 to extend over (e.g., overlap, surround, etc.) the distal end 116 of the proximal outer shaft 115 as the retractable sleeve 135 is withdrawn proximally and transitions to the deployment configuration. For example, a proximal end region of the retractable sleeve 135 may surround the proximal outer shaft 115 (e.g., a distal end region of the proximal outer shaft 115) in both the delivery configuration (FIG. 1) and the deployment configuration (FIG. 4) and the length L2 may be greater than the length of the stent 160 in its radially constrained configuration when constrained within the distal sleeve region 138 in the delivery configuration during delivery to permit a sufficient travel length for the collar 155 to move proximally toward the distal end 116 of the proximal outer shaft 115 as the proximal sleeve region 134 extends over the proximal outer shaft 115 during stent deployment to fully uncover the stent 160. The length L2 of the proximal sleeve region 134 may also be greater than the length L1 of the distal sleeve region 138 to provide a margin of safety that ensures the constrained stent 160 will be fully uncovered when the proximal sleeve region 134 telescopes (e.g., extends over to overlap the proximal outer shaft 115). Additionally, a greater length L2 relative to L1 may assure that the proximal end 132 of the retractable sleeve 135 will remain within the working channel of the endoscope throughout the delivery and deployment of the stent 160. In some instances, the length L1 may be 5 centimeters (cm) or more, 6 cm or more, 7 cm or more, 8 cm or more, 9 cm or more, or 10 cm or more. For example, the length L1 may be about 90 mm (millimeters) to about 130 mm, or about 100 mm to about 120 mm, or about 110 mm in some instances. In some instances, the length L2 may be 20 cm or more, 25 cm or more, 28 cm or more, or 30 cm or more. In some instances, the length L2 may be in the range of 20 cm to 50 cm, in the range of 20 cm to 40, or in the range of 20 cm to 30 cm, for example. In other instances, the length L2 may be about 100 mm to about 140 mm, or about 110 mm to about 130 mm, or about 120 mm. Additionally, as illustrated in FIG. 2, the length L4, measured from a proximal end of the collar 155 to the proximal end of the distal sleeve region 138 proximate the proximal end of the stent 160, may be about 100 mm to about 140 mm, or about 110 mm to about 130 mm, or about 124 mm, in some instances. In some embodiments, the length L1 may be 100 mm to 120 mm, the length L2 may be 110 mm to 130 mm, and the length L4 may be 100 mm to 140 mm. For example, in one embodiment the length L1 may be about 110 mm, the length L2 may be about 120 mm, and the length 14 may be about 124 mm.

In the illustrated example, the proximal sleeve region 134 may surround, i.e., axially overlap, with a distal end region of the proximal outer shaft 115. For instance, referring to FIG. 2, the retractable sleeve 135 is shown having an inner diameter greater than an outer diameter of the proximal outer shaft 115 with the retractable sleeve 135 surrounding the proximal outer shaft 115. In other words, the proximal end 132 of the retractable sleeve 135 may be positioned proximal of the distal end 116 of the proximal outer shaft 115 in both the delivery configuration (FIG. 1) and the deployment configuration (FIG. 4). The extent of acceptable axial overlap may be affected by various factors, including but not limited to, containing the proximal end 132 of the retractable sleeve 135 within the working channel of the endoscope throughout the stent delivery procedure and ensuring that the proximal end 132 of the retractable sleeve 135 surrounds the proximal outer shaft 115 and is axially slidable relative thereto, regardless of whether the retractable sleeve 135 is in the delivery configuration or the deployment configuration. The proximal end 132 of the retractable sleeve 135 may be spaced distally from the handle assembly 104 and unattached thereto, with a length of the proximal outer shaft 115 extending proximally from the proximal end 132 of the retractable sleeve 135 to the second, stationary handle 108 of the handle assembly 104. The length of the proximal outer shaft 115 may be greater than the length of the retractable sleeve 135, and in some instances, the length of the proximal outer shaft 115 may be significantly greater than the length of the retractable sleeve 135, e.g., about 2 or more times greater, or about 3 or more times greater, or about 4 or more times greater, or about 5 or more times greater, or about 6 or more time greater, in some instances.

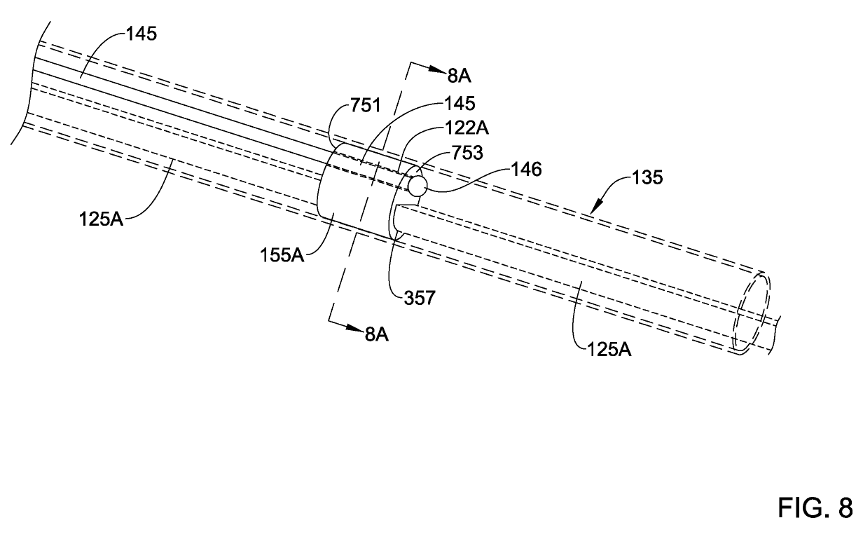

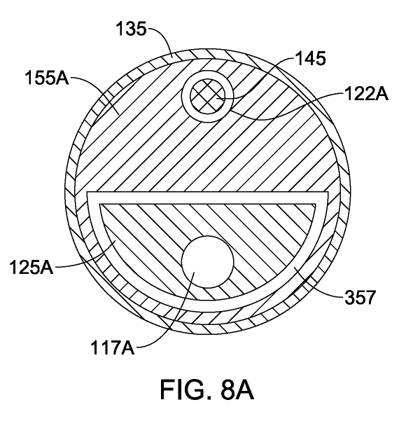

The distal end 146 (shown in FIG. 8) of the actuation shaft 145 may be secured or fixed to a medial region of the retractable sleeve 135 with the proximal sleeve region 134 extending proximal of the medial region and the distal sleeve region 138 extending distal of the medial region. For example, the collar 155 may be positioned within the medial region of the retractable sleeve 135 and the distal end 146 of the actuation shaft 145 secured or fixed thereto. In some instances, the collar 155, such as a tubular collar, may be secured within the lumen of the retractable sleeve 135 with the distal end 146 of the actuation shaft 145 secured to or fixed to (e.g., welded, bonded, etc.) to the collar 155. In some embodiments, the distal end 146 of the actuation shaft 145 may extend through the collar 155 and be positioned distal to the distal end 156 of collar 155 (see also FIG. 8) to secure the distal end 146 of the actuation shaft 145 to the collar 155.

As will be described in greater detail herein, the collar 155 may be configured such that it can axially translate relative to the inner rail 125. In other words, the collar 155 may include a lumen (e.g., opening, aperture) extending longitudinally therethrough which the inner rail 125 may extend through. Accordingly, the collar 155 may axially slide along the inner rail 155 in a distal-to-proximal direction when a proximally-directed force is imparted onto the collar 155. In some examples, the proximally-directed force may be imparted by the actuation shaft 145 onto the collar 155 (e.g., actuating the actuation shaft 145 in a proximal direction may, in turn, actuate the collar 155, and thus the retractable sleeve 135, in a proximal direction.

FIGS. 3A-3B illustrate cross-sectional views taken along line 3-3 of FIG. 1. FIGS. 3A-3B illustrate two different embodiments of the cross-sectional geometry (e.g., cross-sectional shape) of the inner rail 125 described herein. As shown in each of FIGS. 3A and 3B, the inner rail 125 (denoted as 125A or 125B, respectively) may include a non-circular cross-sectional shape (e.g., a non-circular outer periphery).

In the first example, FIG. 3A illustrates that the inner rail 125A may have a generally annular “half-circle” (e.g., semi-circle) cross-sectional shape. Further, the inner rail 125A may include an inner circular opening that forms a guidewire lumen 117A which may permit a guidewire to pass centrally through the guidewire lumen 117A. The guidewire lumen 117A may be axially aligned and in communication with the guidewire lumen 117 extending through the proximal outer shaft 115 as well as a guidewire lumen extending through the distal inner shaft 118.

FIG. 3A illustrates a proximally-directed view taken along line 3-3 of FIG. 1. Accordingly, the proximal outer shaft 115 (shown in dashed lined in FIG. 3A) is visible in FIG. 3A. As described above, the proximal outer shaft 115 may include an actuation shaft lumen 213 within which the actuation shaft 145 may extend. FIG. 3A illustrates that both the actuation shaft lumen 213 and the guidewire lumen 117A of the inner rail 125A may each be offset from a central longitudinal axis of the proximal outer shaft 115, such that the actuation shaft lumen 213 and the actuation shaft 145 extending therein may extend substantially parallel to, but separate from, the guidewire lumen 117A. The actuation shaft 145 may extend along an exterior of the inner rail 125A, and in some cases the actuation shaft 145 may extend generally parallel to the inner rail 125A. This arrangement allows independent movement of the actuation shaft 145 within the actuation shaft lumen 213 while permitting the guidewire lumen 117A to be in communication with the guidewire lumen 217 of the proximal outer shaft 115, as described above with respect to FIGS. 2-3.

FIG. 3B illustrates an alternative embodiment of the cross-sectional shape of the inner rail 125 (shown in FIG. 2) which is analogous to that shown in FIG. 3A, but depicts a different cross-sectional geometry of the inner rail 125 compared to the geometry of the inner rail 125A described with respect to FIG. 3A. In the embodiment of FIG. 3B, the inner rail 125B may include a generally annular body defining a guidewire lumen 117B, while the outer profile may depart from a purely circular shape. The cross-sectional shape of the inner rail 125 shown in FIG. 3B may be characterized by a first lobe 119A and a second lobe 119B formed along an upper region of the inner rail 125, with the lobes 119A, 119B being separated by a recessed valley 120. This contoured geometry may provide distinct surface regions which may engage mating surfaces of the proximal outer shaft 115, while still surrounding the guidewire lumen 117B with structural material. Further, the proximal outer shaft 115 (shown in dashed lined in FIG. 3B) is visible in FIG. 3B. The inner rail 125B may include a guidewire lumen 117B axially aligned and in communication with the guidewire lumen 117 extending through the proximal outer shaft 115 as well as a guidewire lumen extending through the distal inner shaft 118.

As described above, the proximal outer shaft 115 may include an actuation shaft lumen 213 within which the actuation shaft 145 may extend. FIG. 3B illustrates that both the actuation shaft lumen 213 and the guidewire lumen 117B of the inner rail 125B may each be offset from a central longitudinal axis of the proximal outer shaft 115, such that the actuation shaft lumen 213 and the actuation shaft 145 extending therein may extend substantially parallel to, but separate from, the guidewire lumen 117B. The actuation shaft 145 may extend along an exterior of the inner rail 125B, and in some cases the actuation shaft 145 may extend generally parallel to the inner rail 125B. FIG. 3B illustrates that the guidewire lumen 117B and the actuation shaft lumen 213 may be generally aligned with the valley 120 positioned between the first and second lobes 119A, 119B. This configuration may allow the actuation shaft 145 to extend substantially parallel to the guidewire lumen 117B while accommodating the geometry of the inner rail 125B.

FIG. 4 illustrates a side view of the stent delivery system 100 when the retractable sleeve 135 is in the deployment configuration with some components shown in cross-section. More specifically, FIG. 4 illustrates an embodiment of the stent delivery system 100 in which the handle assembly 104 is configured with the first, actuatable handle 106 is positioned proximally to the second, stationary handle 108.

Once the stent 160 has been advanced within the catheter shaft 102 to a target site within the body lumen of the patient, the user may actuate (e.g., move proximally) the first, actuatable handle 106 relative to the second, stationary handle 108 such that the first, actuatable handle 106 moves away from the second, stationary handle 108, for a length L3. Length L3 must be at least of sufficient length to uncover the full axial length of the constrained stent 160. Length L3 is sufficient if, when the first, actuatable handle 106 is moved proximally, the actuation shaft 145 is also retracted proximally by the same length L3.

Further, as a result of retracting the actuation shaft 145 by a length of L3, the retractable sleeve 135, coupled to the actuation shaft 145 via the collar 155, may also be moved proximally by the same length L3 relative to stent 160, in turn exposing and releasing the stent 160 from its radially constrained configuration to a radially expanded, deployed configuration placed at the target site. In sum, actuating the first, actuatable handle 106 relative to the second, stationary handle 108 causes the actuation shaft 145, the collar 155, and the retractable sleeve 135 to proximally move (e.g. translate in a proximal direction) as a unit to uncover stent 160 while the proximal outer shaft 115, the outer reinforcing tube 112, the inner rail 125 and the distal inner shaft 118 remain stationary.

The stent retention bumpers or retainers 164 mounted on the distal inner shaft 118 remain fixedly secure to the distal inner shaft 118 and, therefore, non-moving relative to the retractable sleeve 135 as the retractable sleeve 135 retracts proximally into the deployment configuration. Accordingly, the stent retention bumpers or retainers 164 mounted on the distal inner shaft 118 may act as a stop for the stent 160 as the retractable sleeve 135 is pulled proximally relative to the proximal outer shaft 115 and the inner rail 125, such that the stent 160 does not follow the movement of the retractable sleeve 135 and is deployed on-target.

In conventional stent delivery systems, a singular outer shaft may be secured to a first, actuatable handle and extends distally up to a distal tip of the delivery system to compress and constrain a stent 160 within the outer shaft proximal to the distal tip. In the conventional system, stent deployment is achieved by proximal retraction of the outer shaft relative to an inner shaft that the radially constrained stent surrounds, with the inner shaft secured to a second, stationary handle. However, in stent delivery system 100, because the actuation shaft 145 is internal to the proximal outer shaft 115 and axially moveable relative thereto, the proximal outer shaft 115 may remain stationary. This allows a user to hold onto the stationary components of the stent delivery system 100, such as the proximal outer shaft 115 or the second, stationary handle 108 (to which the proximal outer shaft 115 is fixedly attached) during operation without disturbance to the system's actuation mechanism and consequently preserving the positioning of the stent 160.

The detailed view of FIG. 4 further illustrates the proximal end region 141 of the distal inner shaft 118 attached within a portion of the distal end region 139 of the inner rail 125. It can be appreciated that the inner rail 125 shown in FIG. 4 may embody the cross-sectional geometry shown in either of the examples of the inner rail 125A, 125B shown in FIGS. 3A-3B, respectively. For simplicity, the detailed view shown in FIG. 4 illustrates a distal end region 139 of the example inner rail 125A shown in FIG. 3A attached to a proximal end region 141 of the distal inner shaft 118. The detailed view of FIG. 4 illustrates that the distal end region 139 of the inner rail 125A may include a recessed shoulder which is configured to receive a proximal end region 141 of the distal inner shaft 118 therein. In other words, the proximal end region 141 of the distal inner shaft 118 may be sized to permit it to extend into the distal end region 139 of the inner rail 125A, whereby the proximal end region 141 of the distal inner shaft 118 may engage a recessed shoulder formed within the distal end region 139 of the inner rail 125A. The proximal end region 141 of the distal inner shaft 118 may be bonded (e.g., adhesively bonded) or otherwise secured to the distal end region 139 of the inner rail 125A. Accordingly, FIG. 4 further illustrates that this configuration may permit the guidewire lumen 117A of the inner rail 125A to be in communication with a guidewire lumen 137 of the distal inner shaft 118. It can be appreciated that the guidewire lumen 137 of the distal inner shaft 118 may extend along the entire length of the distal inner shaft 118 and pass through the distal tip 165. In some examples, the proximal end region 141 of the distal inner shaft 118 may be fixedly attached to the distal end region 139 of the inner rail 125A using any suitable attachment technique including gluing or adhesively bonding, thermal bonding, laser welding, ultrasonic welding, mechanical interference fits, over-molding, or combinations thereof.

It can be appreciated that, in some examples, one or more of the proximal outer shaft 115, the inner rail 125, and/or the distal inner shaft 118 may be formed as a unitary structure. For example, one or more of the proximal outer shaft 115, the inner rail 125, and/or the distal inner shaft 118 may be extruded, co-extruded, molded, or otherwise manufactured as a single, integrated, and unified structure. In such examples, the unitary structure may be free from adhesive bonds, welds, or other attachment techniques that would otherwise be used to join discrete components.

As described herein, FIG. 4 illustrates the relationship between the proximal outer shaft 115, the inner rail 125, the collar 155, the actuation shaft 145, and the retractable sleeve 135. In some examples, it may be important for the collar 155 to resist or prevent rotation relative to the inner rail 125 because the collar 155 may be fixedly attached to, or otherwise coupled with, the retractable sleeve 135. During retraction of the retractable sleeve 135 to deploy the stent 160, the retractable sleeve 135 may have a tendency to rotate or spin. If the collar 155 were permitted to rotate relative to the inner rail 125, such rotation could be transferred to the actuation shaft 145, causing the actuation shaft 145 to twist relative to the inner rail 125. This twisting motion may increase friction and deployment forces, potentially raising the forces required for stent deployment to unacceptable levels. Accordingly, it may be desirable to inhibit relative rotation between the collar 155 and the inner rail 125 to minimize deployment forces during retraction of the retractable sleeve 135 to deploy the stent 160.

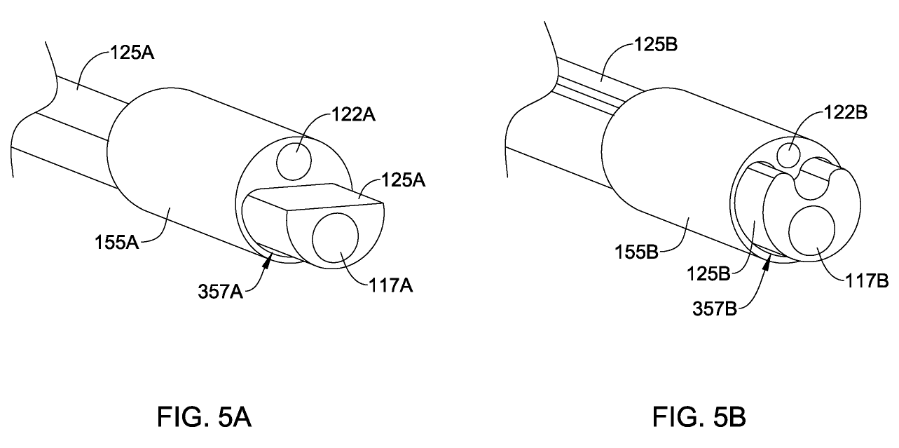

FIG. 5A illustrates a perspective view of the inner rail 125A extending within and through a lumen 357A of the collar 155A. The collar 155A shown in FIG. 5A is one example of the collar 155 described herein with respect to FIGS. 2 and 4. As shown, the inner rail 125A extends longitudinally through the collar 155A such that the collar 155A is movable (e.g., axially translatable, slidable) relative to the inner rail 125A in an axial direction. Thus, the collar 155A may axially slide along the inner rail 125A during deployment of the stent 160. FIG. 5A further illustrates a guidewire lumen 117A described herein extending within the inner rail 125A.

As can be appreciated from FIG. 5A, the lumen 357A defines a geometry that corresponds (e.g., matches, mates with) to the cross-sectional geometry of the inner rail 125A. For example, the lumen 357A may be non-circular and configured to slidably receive the inner rail 125A also having a non-circular outer periphery, thereby keying the inner rail 125A with the collar 155A. Because the cross-sectional geometry of the lumen 357A is not completely circular, the inner rail 125A may mechanically constrain the collar 155A, thereby inhibiting relative rotational movement between the collar 155A and the inner rail 125A. In this manner, the non-circular geometry of the lumen 357A and the non-circular geometry of the inner rail 125A prevents or reduces rotational spinning of the collar 155A and the actuation shaft 145 relative to the inner rail 125A while still permitting axial translation of the collar 155A along the inner rail 125A.

As further shown in FIG. 5A, the collar 155A may define an additional lumen 122A configured to receive the actuation shaft 145 (shown in FIG. 4).

FIG. 5B illustrates a perspective view of the inner rail 125B extending within a lumen 357B of a collar 155B. The collar 155B shown in FIG. 5B is another example of the collar 155 described herein with respect to FIGS. 2 and 4. As shown, the inner rail 125B extends longitudinally through the collar 155B such that the collar 155B is movable (e.g., translatable, slidable) relative to the inner rail 125B in an axial direction. FIG. 5B further illustrates a guidewire lumen 117B described herein extending within the inner rail 125B.

As can be appreciated from FIG. 5B, the lumen 357B defines a geometry that corresponds (e.g., matches, mates with) to the cross-sectional geometry of the inner rail 125B. For example, the lumen 357B may be non-circular and configured to slidably receive the inner rail 125B also having a non-circular outer periphery, thereby keying the inner rail 125B with the collar 155B. Because the cross-sectional geometry of the lumen 357B is not completely circular, the inner rail 125B may mechanically constrain the collar 155B, thereby inhibiting relative rotational movement between the collar 155B and the inner rail 125B. In this manner, the non-circular geometry of the lumen 357B and the non-circular geometry of the inner rail 125B prevents or reduces rotational spinning of the collar 155B and the actuation shaft 145 relative to the inner rail 125B while still permitting axial translation of the collar 155B along the inner rail 125B.

As further shown in FIG. 5B, the collar 155B may define an actuation lumen 122B configured to receive the actuation shaft 145 (shown in FIG. 4).

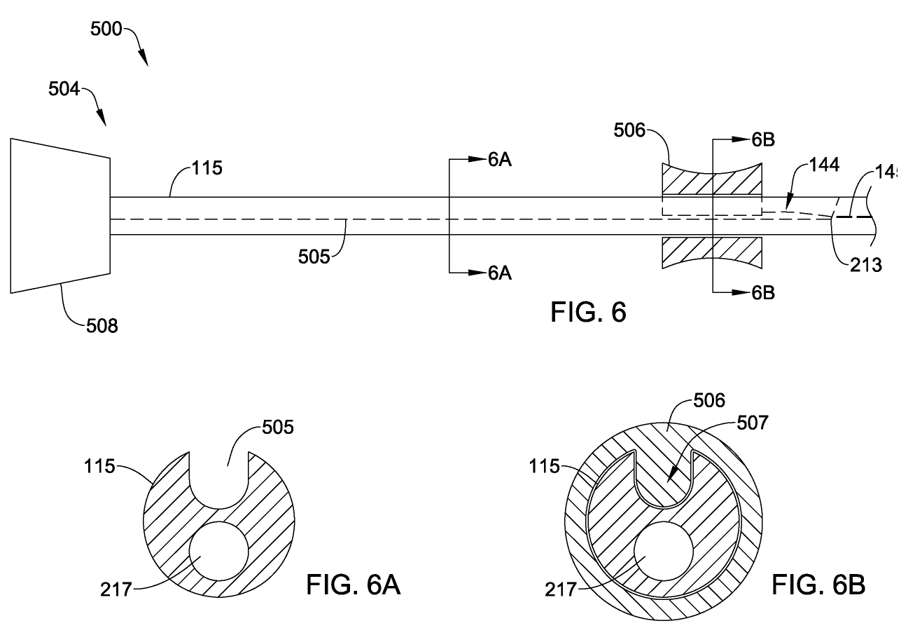

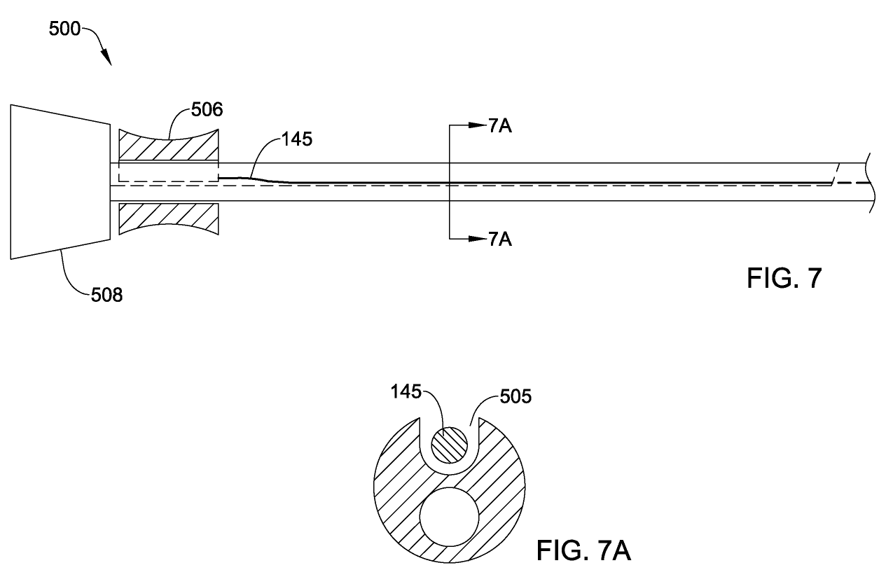

FIGS. 6 and 7 show a stent delivery system 500 with an alternative handle assembly 504 configuration, wherein the first, actuatable handle 506, which is actuatable, is positioned distal to the second, stationary handle 508. FIG. 6 shows a side view of the handle assembly 504 of the stent delivery system 500 when the retractable sleeve 135 (shown in FIG. 2) is in the delivery configuration as shown in FIG. 1, whereas FIG. 7 shows a side view of the handle assembly 504 of the stent delivery system 500 when the retractable sleeve 135 is in the deployment configuration as shown in FIG. 4. The proximal end 144 of the actuation shaft 145 may be fixed to the first, actuatable handle 506 and extend distally therefrom. Likewise, the proximal end of the proximal outer shaft 115 may be fixed to the second, stationary handle 508 and extend distally therefrom.

In this embodiment, the proximal outer shaft 115 may include a longitudinal channel 505, within which the actuation shaft 145 may be positioned. The longitudinal channel 505 may run along a portion of the length of the proximal outer shaft 115 to serve as a pathway for the proximal end region 144 of the actuation shaft 145 to travel, allowing the proximal end region 144 of actuation shaft 145 to exit the actuation shaft lumen 213 of the proximal outer shaft 115, travel along the exterior of the proximal outer shaft 115, and couple to the first, actuatable handle 506 for operation. Additionally, the longitudinal channel 505 helps keep the actuation shaft 145 properly guided and contained, preventing inadvertent movement or interference that could lead to inaccuracies during deployment.

FIG. 6A shows a cross-sectional view of the proximal outer shaft 115 taken along line 6A-6A of FIG. 6. The proximal outer shaft 115 includes the guidewire lumen 217 and the longitudinal channel 505, which is in communication with the actuation shaft lumen 213 (shown in FIG. 2) extending distally of the longitudinal channel 505 through the proximal outer shaft 115 to the distal end of the proximal outer shaft 115. In this example, the longitudinal channel 505 is illustrated with a U-shaped cross-section opening laterally to a periphery of the proximal outer shaft 115. In some examples, the cross-sectional shape may be specifically selected to reflect the cross-sectional shape of the actuation shaft 145. In this example, a U-shaped cross-section may be selected for an actuation shaft 145 that is a rounded pull wire. However, a match in the cross-sectional shapes of the longitudinal channel 505 and the actuation shaft 145 is not required.

FIG. 6B illustrates a cross-sectional view of the proximal outer shaft 115 taken along line 6B-6B of FIG. 6. FIG. 6B illustrates the actuatable handle 506 extending around the outer surface of the proximal outer shaft 115. FIG. 6B further illustrates that the actuatable handle 506 may include a projection 507 extending radially inward from an inwardly-facing surface of the actuatable handle 506 which may be configured to extend into the longitudinal channel 505 of the proximal outer shaft 115. Further, it can be appreciated that the cross-sectional shape of the projection 507 may match that cross-sectional shape (e.g., the U-shaped cross-section) of the longitudinal channel 505. Accordingly, when the projection 507 extends into the longitudinal channel 505, the actuatable handle 506 may be prevented from rotating or spinning along the outer surface of the proximal outer shaft 115 while permitting the actuatable handle 506 to longitudinally translate along the proximal outer shaft 115. Additionally, FIG. 6 illustrates that the proximal end region 144 of the actuation shaft 145 may be attached to the projection 507.

To deploy the stent 160, the user may actuate the first, actuatable handle 506 (i.e., move proximally) relative to the second, stationary handle 508 such that the first, actuatable handle 506 moves towards the second, stationary handle 508. Actuation of the first, actuatable handle 506 actuates the actuation shaft 145 proximally relative to the proximal outer shaft 115, which in turn withdraws the retractable sleeve 135 (shown in FIG. 2) proximally over and relative to the distal end of the proximal outer shaft 115 into the delivery configuration, resulting in deployment of the stent 160 in a similar manner to the stent delivery system 100 of FIG. 4.

The longitudinal channel 505 may have a length sufficient to accommodate the full length of movement required by the actuation shaft 145 during deployment. In the illustrated example, the length of the longitudinal channel 505 is such that the first, actuatable handle 506 and the second, stationary handle 508 abut against each other once in the deployment configuration. However, in other examples, the first, actuatable handle 506 and the second, stationary handle 508 may not abut in the deployment configuration because the retractable sleeve 135 (shown in FIG. 2) has already traveled a sufficient length to release the stent 160 from its radially constrained configuration.

FIG. 7A shows a cross-sectional view of the proximal outer shaft 115 taken along line 7A-7A of FIG. 7, showing the actuation shaft 145 positioned in the longitudinal channel 505. In this example, the actuation shaft 145 is a rounded pull wire, however the actuation shaft 145 may differ in shape so long as it is able to move through the track created by the longitudinal channel 505.

FIG. 8 illustrates a perspective view of the stent delivery system 100 showing the inner rail 125A extending longitudinally through a lumen 357 (shown in FIG. 8A) of the collar 155A. As described herein, the collar 155A may be configured to move axially (e.g., translate) relative to and along the inner rail 125A, however, the mating non-circular geometry of the inner rail 125A and the lumen 357 of the collar 155A may inhibit relative rotation therebetween.

FIG. 8 further illustrates the actuation shaft 145 extending through the actuation lumen 122A (shown in FIG. 8A). A distal end of the actuation shaft 145 may include a tip, ball, or other enlarged portion 146 that may be attached to the distal end region of the actuation shaft 145 or formed as a unitary portion thereof. In some examples, the enlarged portion 146 may comprise an enlarged protuberance, such as a welded bead, swaged end, or other radially enlarged structure. Further, the enlarged portion 146 may have an outer dimension greater than a diameter of the actuation lumen 122A such that the enlarged portion 146 may be prevented from passing proximally through the actuation lumen 122A. Accordingly, a proximal retraction of the actuation shaft 145 may cause the enlarged portion 146 to engage a portion of a distally-facing surface 753 of the collar 155A which defines the opening of the actuation lumen 122A and thereby apply a proximally-directed force to the collar 155A. This force may cause the collar 155A to translate in a distal-to-proximal direction. In some examples, a retractable sleeve 135 may be fixedly attached to the collar 155A such that proximal translation of the collar 155A may result in corresponding proximal movement of the retractable sleeve 135.

Additionally, in some examples, a distal end of the actuation shaft 145 may be coupled to the collar 155A using one or more different configurations than that illustrated in FIG. 8. For example, the distal end of the actuation shaft 145 may be attached to, engaged with, or otherwise secured to other portions of the collar 155A. In some examples, the distal end of the actuation shaft 145 may be attached to or engage a proximal-facing surface 751 of the collar 155A. In yet other examples, the distal end of the actuation shaft 145 may be attached to or engaged to the collar 155A within the actuation lumen 122A. In such embodiments, movement of the actuation shaft 145 in a proximal direction may transmit forces to the collar 155A to cause corresponding axial translation of the collar 155A and the retractable sleeve 135 as described herein.

In some embodiments, the actuation shaft 145 may have a reduced diameter from the portion extending between the proximal surface 751 of the collar 155A and the distal surface 753 of the collar 155A relative to a diameter of the actuation shaft 145 extending proximally from the proximal surface 751 of the collar 155A. Accordingly, the reduced diameter of the actuation shaft 145 may form a shoulder at the proximal surface 751, and thus the shoulder may abut the proximal surface 751. The shoulder of the actuation shaft 145 may prevent distal movement of the actuation shaft 145 relative to the collar 155A, thus enabling distal actuation of the collar 155A, and thus distal actuation of the retractable sleeve 135 via distal movement of the actuation shaft 145.

In some examples, after deployment of the stent 160, it may be desirable to advance the retractable sleeve 135 in a proximal-to-distal direction such that the distal end 136 (shown in FIG. 1) of the retractable sleeve 135 engages or contacts the distal tip 165 (shown in FIG. 1), thereby reducing the likelihood that a portion of the distal tip 165 contacts the deployed stent 160 as the stent delivery system 100 is withdrawn from a body lumen.

FIG. 8A illustrates a cross-sectional view taken along line 8A–8A of FIG. 8. FIG. 8A illustrates the inner rail 125A extending within the lumen 357 of the collar 155A. As described herein, the lumen 357 includes a geometry that matches the outer profile of the inner rail 125A. FIG. 8A further illustrates the guidewire lumen 117A extending through the inner rail 125A which may be configured to permit a guidewire (not shown) to extend within. As described herein, the shape (e.g., non-circular shape, non-circular outer periphery) of the inner rail 125A and corresponding shape (e.g., non-circular shape, non-circular periphery) of the lumen 357 through which it extends may work cooperatively to inhibit relative rotational movement between the collar 155A and the inner rail 125A, while permitting axial translation of the collar 155A along the inner rail 125A. The collar 155A may further define an actuation lumen 122A through which the actuation shaft 145 extends. Further, the retractable sleeve 135 is shown disposed around, and fixedly attached to, the collar 155A such that movement of the collar 155A results in corresponding movement of the retractable sleeve 135.

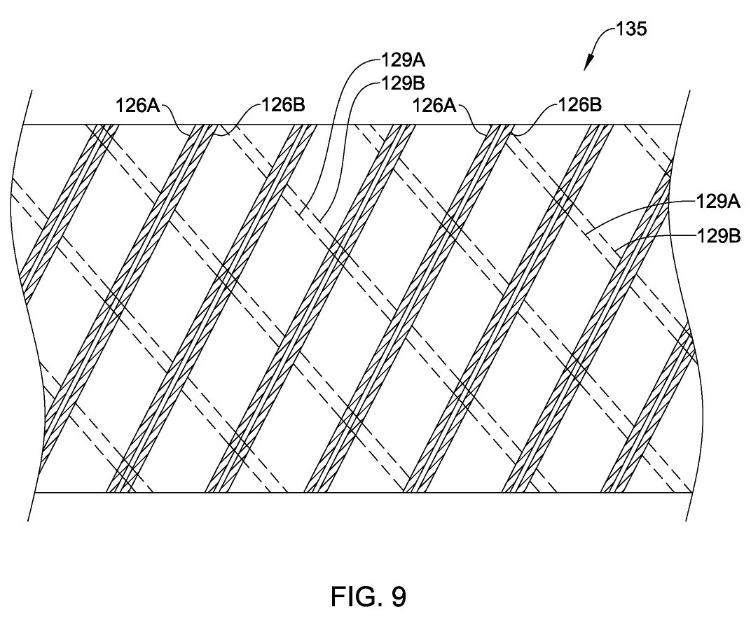

FIG. 9 illustrates that the retractable sleeve 135 may include a braided or other similar interwoven construction. As illustrated in FIG. 9, the retractable sleeve 135 may include a wire component formed of at least one metallic wire, such as from a first wire 126A positioned adjacent to a second wire 126B, whereby the first and second wires 126A, 126B may be wound, braided, interwoven, etc. in a helical arrangement extending along the length of the retractable sleeve 135. In other examples, the wire component may include only a single wire versus the two-wire arrangement shown in FIG. 9. In yet other examples, the wire component may include more than two wires. The first and/or second wires 126A, 126B may be formed of a metallic material, such as stainless steel, tungsten, or nitinol. All the first and/or second wires 126A, 126B may extend in a first helical direction around the retractable sleeve 135.

FIG. 9 illustrates that the retractable sleeve 135 may further include a polymer component formed of at least one polymeric filament, such as from a first filament 129A positioned adjacent to a second filament 129B, whereby the first and second filaments 129A, 129B may be wound, braided, overlaid on, or interwoven with the wire component including the first and/or second wires 126A, 126B. FIG. 9 illustrates that the polymer filaments 129A, 129B may be wound in a second, opposite helical direction relative to the first and second wires 126A, 126B. Accordingly, all of the first and/or second wires 126A, 126B, which may be formed of a metallic material) may extend in a first helical direction, and all of the polymer filaments 129A, 129B may extend in a second helical direction, opposite the first helical direction. In some embodiments, the polymer filaments 129A, 129B may be incorporated into the retractable sleeve 135 (e.g., interwoven with the first and/or second wires 126A, 126B) via braiding, weaving, knitting, wrapping, coiling, or other suitable construction techniques. In some examples, the first wire 126A, second wire 126B, the first polymer filament 129A and the second polymer filament 129B may be braided using a 50 pic diamond pattern using a 32-carrier braiding machine. For instance, 16 metal wires 126A, 126B may be loaded onto 16 carriers of the braiding machine, while 16 polymer filaments 129A, 129B may be loaded onto 16 additional carriers of the braiding machine in order to braid the polymer filaments with the metal wires to form the braided reinforcement structure of the retractable sleeve 135.

In some examples, each of the first and second wires 126A, 126B may have an outer diameter of about 0.015 mm (millimeters) to about 0.35 mm, or about 0.020 mm to about 0.30 mm, or about 0.022 mm to about 0.27 mm, or about .025 mm to about 0.25 mm. In some examples, the polymer filaments 129A, 129B may have an outer diameter of about 0.025 mm to about 0.85 mm, or about 0.035 mm to about 0.75 mm, or about 0.045 mm to about 0.65 mm, or about .065 mm to about 0.80 mm, or about 0.070 mm to about 0.80 mm. In other examples, the polymer filaments 129A, 129B may each be a flat filament including a square or rectangular cross-sectional “bar” shape, whereby the width of the rectangular cross-sectional shape may be about 0.025 mm to about 0.510 mm and the height of the rectangular cross-sectional shape may be about 0.0125 mm to about 0.275 mm.

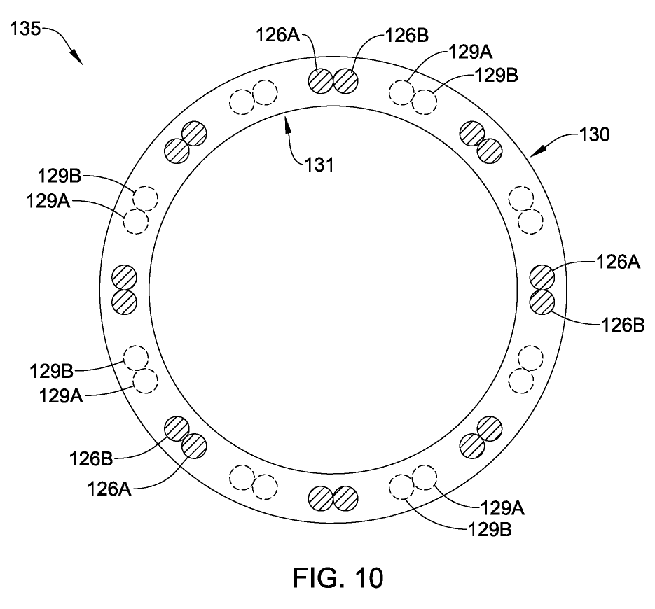

FIG. 10 illustrates a cross-sectional view of the example retractable sleeve 135 shown in FIG. 10. As shown in FIG. 10, the retractable sleeve 135 may include a first layer 130 (which may also be referred to as an outer layer and may define a base material of the retractable sleeve 135) and a second layer 131 (e.g., an inner layer) positioned radially inward of the first layer 130. In some embodiments, the first layer 130 may be formed from a polymeric material such as a polyether block amide (PEBA) such as Pebax^{®}, thermoplastic polyurethane (TPU), such as Pellethane^{®}, or another suitable polymer material. In some embodiments, the second layer 131 may be formed from a polymeric material such as a polytetrafluoroethylene (PTFE) such as Tecoflex^{®} or another suitable polymer material.

Additionally, FIG. 10 further illustrates the first wire 126A, second wire 126B, the first polymer filament 129A and the second polymer filament 129B may be embedded within or positioned between the first layer 130 and the second layer 131. In some embodiments, the polymer filaments 129A, 129B may be formed from a material selected to visually blend with the material of the first layer 130. For example, the polymer filaments 129A, 129B may be formed from a polyamide or other suitable material which has a refractive index that is similar to, or substantially matches, the refractive index of the material used to construct the first layer 130. In some examples, the refractive index of the polymer filaments 129A, 129B and the refractive index of the material used to construct the first layer 130 may be within about 0.3 or less from each other, or about 0.25 or less from each other, or about 0.20 or less from each other, or about 0.15 or less from each other, or about 0.10 or less from each other, or about 0.05 or less from each other, or about 0.025 or less from each other, or between 0.01 and 0.02 from each other, or about 0.01 from each other. Accordingly, the polymer filaments 129A, 129B may visually blend with the material used to construct the first layer 130. Further, such visual blending may improve visualization through the retractable sleeve 135, which may be important for differentiating the stent 160 (shown in FIG. 2), which may have a braided wire construction, from the braided sleeve 135 during use. Improved visualization through the retractable sleeve 135 may also allow better visualization of underlying components, such as the stent retention bumper or retainer 164, positioned beneath the retractable sleeve 135, which may be important for accurate positioning of the stent 160 within a target site.

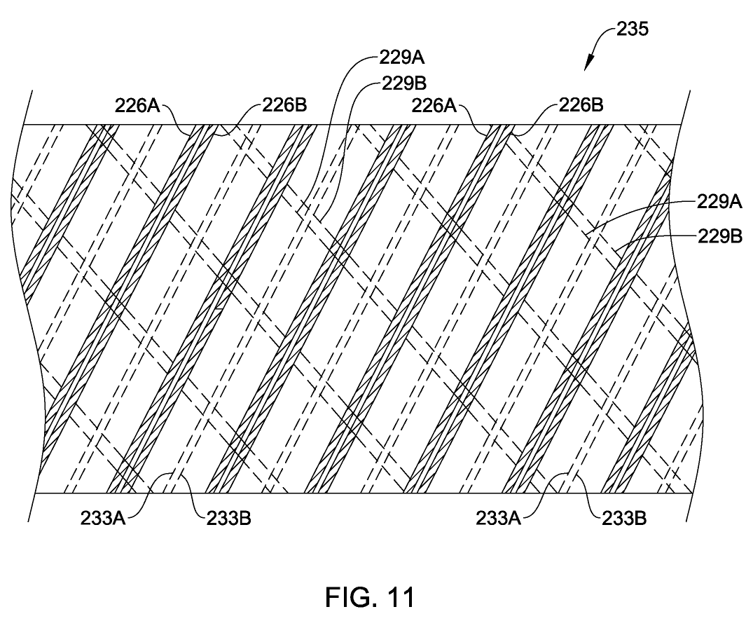

FIG. 11 illustrates another example construction of the retractable sleeve 235 similar in form and function to the example retractable sleeve 135 described in FIGS. 9-10. As illustrated in FIG. 11, the retractable sleeve 235 may include a wire component formed of at least one metallic wire, such as formed from a first wire 226A positioned adjacent to a second wire 226B, whereby the first and second wires 226A, 226B may be wound, braided, interwoven, etc. in a helical arrangement extending along the length of the retractable sleeve 235. In other examples, the wire component may include only a single wire versus the two-wire arrangement shown in FIG. 11. In yet other examples, the wire component may include more than two wires. The first and/or second wires 226A, 226B may be formed of a metallic material, such as stainless steel, tungsten, or nitinol. All the first and/or second wires 226A, 226B may extend in a first helical direction around the retractable sleeve 235.

FIG. 11 illustrates that the retractable sleeve 235 may further include a first polymer component formed of at least one polymeric filament, such as from a first filament 229A positioned adjacent to a second filament 229B, whereby the first and second filaments 229A, 229B may be wound, braided, overlaid on, or interwoven with the wire component including the first and/or second wires 226A, 226B. Further, FIG. 11 illustrates that the retractable sleeve 235 may further include a second polymer component formed from a third filament 233A positioned adjacent to a fourth filament 233B, whereby the third and fourth filaments 233A, 233B may be wound, braided, overlaid on, or interwoven with the wire component including the first and/or second wires 226A, 226B and the first polymer component including the first and second filaments 229A, 229B. FIG. 11 illustrates that the polymer filaments 233A, 233B may be wound in the same direction as the first and/or second wires 226A, 226B and in a second, opposite helical direction relative to the first and second filaments 229A, 229B. Accordingly, all of the first and/or second wires 226A, 226B (which may be formed of a metallic material) and the third and fourth filaments 233A, 233B, may extend in a first helical direction, and all of the polymer filaments 229A, 229B may extend in a second helical direction, opposite the first helical direction.

In some embodiments, the polymer filaments 229A, 229B, 233A, 233B may be incorporated into the retractable sleeve 235 (e.g., interwoven with the first and/or second wires 226A, 226B) via braiding, weaving, knitting, wrapping, coiling, or other suitable construction techniques. In some examples, the first wire 226A, second wire 226B, the first polymer filament 229A, the second polymer filament 229B, the third polymer filament 233A and the fourth polymer filament 233B may be braided using a 50 pic diamond pattern using a 32-carrier braiding machine. For instance, 16 metal wires 226A, 226B may be loaded onto 16 carriers of the braiding machine, while the 8 polymer filaments 229A, 229B may be loaded onto 8 additional carriers and the 8 polymer filaments 233A, 233B may be loaded onto 8 additional carriers of the braiding machine in order to braid the polymer filaments with the metal wires to form the braided reinforcement structure of the retractable sleeve 235.

In some examples, each of the first and second wires 226A, 226B may have an outer diameter of about 0.015 mm (millimeters) to about 0.35 mm, or about 0.020 mm to about 0.30 mm, or about 0.022 mm to about 0.27 mm, or about .025 mm to about 0.25 mm. In some examples, the polymer filaments 229A, 229B, 233A, 233B may have an outer diameter of about 0.025 mm to about 0.85 mm, or about 0.035 mm to about 0.75 mm, or about 0.045 mm to about 0.65 mm, or about .065 mm to about 0.80 mm, or about 0.070 mm to about 0.80 mm. In other examples, the polymer filaments 229A, 229B, 233A, 233B may be flat filaments including a square or rectangular cross-sectional “bar” shape, whereby the width of the rectangular cross-sectional shape may be about 0.025 mm to about 0.510 mm and the height of the rectangular cross-sectional shape may be about 0.0125 mm to about 0.275 mm.

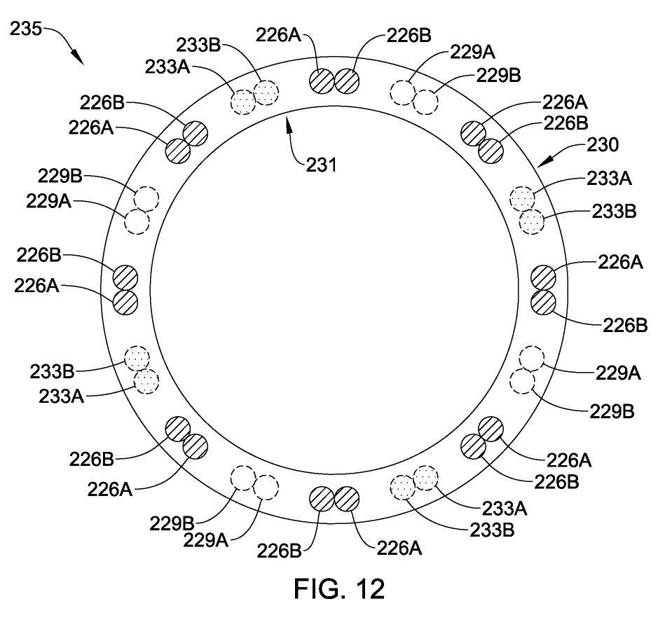

FIG. 12 illustrates a cross-sectional view of the example retractable sleeve 235 shown in FIG. 11. As shown in FIG. 12, the retractable sleeve 235 may include a first layer 230 (which may also be referred to as an outer layer and may define a base material of the retractable sleeve 235) and a second layer 231 (e.g., an inner layer) positioned radially inward of the first layer 230. In some embodiments, the first layer 230 may be formed from a polymeric material such as a polyether block amide (e.g., Pebax®), thermoplastic polyurethane (TPU), such as Pellethane®, or another suitable polymer material. In some embodiments, the second layer 231 may be formed from a polymeric material such as a polytetrafluoroethylene (e.g., Tecoflex®) or another suitable polymer material.

Additionally, FIG. 12 further illustrates the first wire 226A, second wire 226B, the first polymer filament 229, the second polymer filament 229B, the third polymer filament 233A and the fourth polymer filament 233B may be embedded within or positioned between the first layer 230 and the second layer 231. In some embodiments, the polymer filaments 229A, 229B, 233A, 233B may be formed from a material selected to visually blend with the material of the first layer 230. For example, the polymer filaments 229A, 229B, 233A, 233B may be formed from a polyamide or other suitable material which has a refractive index that is similar to, or substantially matches, the refractive index of the material used to construct the first layer 230. In some examples, the refractive index of the polymer filaments 229A, 229B, 233A, 233B and the refractive index of the material used to construct the first layer 230 may be within about 0.3 or less from each other, or about 0.25 or less from each other, or about 0.20 or less from each other, or about 0.15 or less from each other, or about 0.10 or less from each other, or about 0.05 or less from each other, or about 0.025 or less from each other, or between 0.01 and 0.02 from each other, or about 0.01 from each other. Accordingly, the polymer filaments 229A, 229B, 233A, 233B may visually blend with the material used to construct the first layer 230. Further, such visual blending may improve visualization through the retractable sleeve 235, which may be important for differentiating the stent 260 (shown in FIG. 2), which may have a braided wire construction, from the braided sleeve 235 during use. Improved visualization through the retractable sleeve 235 may also allow better visualization of underlying components, such as the stent retention bumper or retainer 264, positioned beneath the retractable sleeve 235, which may be important for accurate positioning of the stent 260 within a target site.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent delivery system comprising:
a handle;
a catheter shaft extending distally from the handle, the catheter shaft including a proximal outer shaft, an inner rail coupled to the proximal outer shaft and a distal inner shaft coupled to the inner rail;
a collar disposed along and translatable relative to the inner rail, the collar defining a first lumen configured to permit the inner rail to extend therethrough; and
a radially expandable stent disposed along a portion of the distal inner shaft;
wherein the first lumen has a first cross-sectional shape which mates with a second cross-sectional shape of the inner rail such that relative rotational movement between the collar and the inner rail is inhibited while axial translation of the collar relative to the inner rail is permitted.

2. The stent delivery system of claim 1, wherein the first cross-sectional shape and the second cross-sectional shape are non-circular.

3. The stent delivery system of claim 1, wherein the first cross-sectional shape and the second cross-sectional shape are semi-circular.

4. The stent delivery system of claim 1, wherein the first cross-sectional shape and the second cross-sectional shape include at least one flat surface.

5. The stent delivery system of claim 1, wherein the first cross-sectional shape and the second cross-sectional shape include a first lobe, a second lobe and a valley separating the first lobe and the second lobe.

6. The stent delivery system of claim 1, wherein the inner rail includes a guidewire lumen which is in communication with a guidewire lumen of the proximal outer shaft.

7. The stent delivery system of claim 1, further comprising a retractable sleeve attached to the collar.

8. The stent delivery system of claim 7, wherein the stent is radially constrained between the distal inner shaft and the retractable sleeve in a delivery configuration.

9. The stent delivery system of claim 8, further comprising an actuation shaft coupled to the collar, wherein actuation of the actuation shaft axially translates both the collar and the retractable sleeve relative to the inner rail.

10. The stent delivery system of claim 9, wherein the collar includes an actuation shaft lumen configured to permit at least a portion of the actuation shaft to extend therein.

11. The stent delivery system of claim 10, wherein the axial translation of the retractable sleeve permits deployment of the stent.

12. The stent delivery system of claim 11, wherein the mating cross-sectional shape of the first lumen and the inner rail inhibits twisting of the actuation shaft with the inner rail.

13. The stent delivery system of claim 12, wherein the retractable sleeve includes a braided structure including a first wire extending in a first helical direction along a length of the retractable sleeve, and wherein the retractable sleeve further includes a polymer filament wound in a second helical direction opposite the first helical direction, and wherein the first wire and the polymer filament are embedded within a base material used to construct the retractable sleeve.

14. The medical device of claim 13, wherein the polymer filament has a refractive index similar to a refractive index of the base material.

15. A stent delivery system comprising:
a handle;
a catheter shaft extending distally from the handle, the catheter shaft including a proximal outer shaft, an inner rail coupled to the proximal outer shaft and extending distally therefrom, and a distal inner shaft coupled to the inner rail and extending distally therefrom;
a collar disposed along and axially translatable relative to the inner rail, the collar defining a first lumen configured to permit the inner rail to extend therethrough;
a retractable sleeve attached to the collar, the retractable sleeve including a base material and at least one reinforcing filament having a refractive index similar to a refractive index of the base material; and
a radially expandable stent positioned between the retractable sleeve and the distal inner shaft in a delivery configuration;
wherein the first lumen has a first cross-sectional shape which mates with a second cross-sectional shape of the inner rail such that relative rotational movement between the collar and the inner rail is inhibited while axial translation of the collar relative to the inner rail is permitted.

16. The stent delivery system of claim 15, wherein the first cross-sectional shape and the second cross-sectional shape are non-circular.

17. The stent delivery system of claim 15, wherein the retractable sleeve includes at least one wire interwoven with the at least one reinforcing filament, wherein the at least one wire extends in a first helical direction and the at least one reinforcing filament extends in a second helical direction, wherein the second helical direction is opposite the first helical direction.

18. The stent delivery system of claim 17, wherein the at least one wire is formed of stainless steel, the at least one reinforcing filament is formed of polyamide, and the base material is a polyether block amide or a thermoplastic polyurethane.

19. A stent delivery system comprising:
a handle;
a catheter shaft extending distally from the handle, the catheter shaft including a proximal outer shaft, an inner rail fixed relative to the proximal outer shaft and extending distally therefrom, and a distal inner shaft fixed relative to the inner rail and extending distally therefrom;
a collar disposed along and axially translatable relative to the inner rail, the collar defining a first lumen configured to permit the inner rail to extend therethrough;
a retractable sleeve attached to the collar, the retractable sleeve including a base material and at least one reinforcing filament;
a radially expandable stent positioned between the retractable sleeve and the distal inner shaft in a delivery configuration; and
an actuation shaft including a first end coupled to the handle and a second end coupled to the collar, wherein actuation of the actuation shaft is configured to axially translate the retractable sleeve in a distal-to-proximal direction to deploy the stent;
wherein the first lumen has a first cross-sectional shape which mates with a second cross-sectional shape of the inner rail such that relative rotational movement between the collar and the inner rail is inhibited while axial translation of the collar relative to the inner rail is permitted.

20. The stent delivery system of claim 19, wherein the first cross-sectional shape and the second cross-sectional shape are non-circular.
